(19) United States
(12) Patent Application Publication
(10) Pub. No.: US 2013/0079596 A1
(43) Pub. Date: Mar. 28, 2013

(54) DYNAMIC SURGICAL FLUID SENSING

(71) Applicant: Todd Edward Smith, Hopedale, MA (US)

(72) Inventor: Todd Edward Smith, Hopedale, MA (US)

(21) Appl. No.: 13/242,370

(22) Filed: Sep. 23, 2011

(51) Int. Cl. A61B 1/12

(52) U.S. Cl. 600/118

(57) ABSTRACT

A dynamic sensing method and apparatus employs microelectromechanical systems (MEMS) and nanoelectromechanical (NEMS) surgical sensors for gathering and reporting surgical parameters of fluid flow and other characteristics of the surgical field. A medical device employs or affixes the surgical sensor in a fluid flow path of the fluids transferred during the surgical procedure. The surgical procedure disposes the medical device in the surgical field responsive to the fluid flow, such as in a cannula or other endoscopic instrument inserted in a surgical void defined or utilized by the surgical procedure. The reduced size of the surgical sensor allows nonintrusive placement in the surgical field, such that the sensor does not interfere with or adversely affect the flow of the fluid it is intended to measure. The reduced size is also favorable to manufacturing costs and waste for single use and disposable instruments which are discarded after usage on a patient.

### BACKGROUND

Design and development of electronics has steadily been following a downsizing trend ever since Gordon Moore, cofounder of Intel® corporation, suggested in 1965 that the transistor density (hence computing power) of a given chip area doubles roughly every 24 months, in a somewhat prophetic assertion that has become widely known as “Moore's Law.” Medical devices and apparatus are no exception to the trend of electronics miniaturization. Microelectronics are often employed as sensors for providing diagnostic feedback on routine patient status, such as for sensing pulse, oxygen saturation, body temperature, and fetal vitals during childbirth.

During surgical procedures, sensing often extends to the transfer of fluids between a patient and medical apparatus. Various fluid exchanges are often involved during surgery, such as blood, saline, and medications, to name several, for such purposes as fluid loss compensation, irrigation of the surgical field, and automated medication delivery. Electronics for sensing fluidic parameters are often employed for sensing patient attributes such as fluid pressure, flow and temperature, for example.

### SUMMARY

A dynamic sensing method and apparatus employs microelectromechanical systems (MEMS) and nanoelectromechanical (NEMS) surgical sensors for gathering and reporting surgical parameters of fluid flow and other characteristics of the surgical field. A medical device employs or affixes the surgical sensor on or about a fluid flow path of the fluids transferred during the surgical procedure. The surgical procedure disposes the medical device in the surgical field responsive to the fluid flow, such as in a cannula or other endoscopic instrument inserted in a surgical void defined or utilized by the surgical procedure. The reduced size of the surgical sensor allows nonintrusive placement in the surgical field, such that the sensor does not interfere with or adversely affect the flow of the fluid it is intended to measure. The reduced size is also favorable to manufacturing costs and waste for single use and disposable instruments which are discarded after usage on a single patient. Surgical parameters such as pressure, flow and temperature are measured at the surgical site rather than indirectly via remote fluid sources, rendering a more accurate reading of the surgical parameters while responsive to dynamic conditions immeasurable with conventional RFID devices.

In a surgical environment, various fluids are often exchanged throughout the course of a surgical procedure (operation). These fluids include blood, saline, medications, irrigation waste, anesthetic gas, oxygen, and others. Monitoring and retrieving surgical parameters related to the various fluids provides diagnostic feedback to surgeons and medical staff. During an endoscopic surgical procedure, for example, a fluid management system often provides saline to an internal surgical site for irrigating and expanding the surgical field.

In configurations disclosed below, a surgical fluid management system employs MEMS or NEMS (Microelectromechanical or Nanoelectromechanical systems) sensors to provide performance data and statistics to the processor of the fluid management system during a surgical procedure for employing the sensor data in logic instructions responsive to the sensors. It is further beneficial if such sensors are small and disposable, to permit unobtrusive placement and to mitigate waste and cost for non-reusable surgical equipment. The surgical fluid data is typically dynamic and therefore amenable to regular monitoring and response. For example, a valuable but often underutilized data item is accurate determination of in-joint fluid data to allow this information to be utilized during a surgical procedure. Configurations of the proposed approach allow utilization of such data by placing a MEMS sensor within the joint via attachment to other surgical instrumentation or as a dedicated device.

Configurations herein are based, in part, on the observation that conventional approaches employ RFID (Radio Frequency Identification) tags on surgical tools and equipment for tracking during a surgical procedure. While RFIDs can be fabricated to be small and passive (i.e. externally powered by the triggering signal), computation and execution power is limited. Unfortunately, therefore, conventional approaches to device interconnection suffer from the shortcoming that response is typically limited to identification of the device or instrument on which the RFID is affixed, and information other than identity is unavailable, due to limited computational ability that may be encoded on an RFID.

Accordingly, configurations herein substantially overcome the above described shortcomings by providing an unobtrusive sensor device disposed in the surgical field for direct sensing of surgical parameters as well as transmission capabilities for communicating sensed parameters to a fluid management system. In contrast to conventional approaches, which utilize non-invasive (external) sensors or transducers integrated into the fluid management system, the proposed approach employs sensors disposed at the surgical site. Direct, invasive evaluation provided by the proposed approach allows accurate sensor readings of pressure, flow and other measurements, providing better accuracy than, for example, indirect transducer measurements from a tube set attached to the fluid management system. The use of MEMS and NEMS devices permits placement within the surgical site, such as in a knee joint between articulated skeletal members, and a wireless interface allows transmission of the fluid data without interfering with other aspects or instruments of the surgical procedure.

In further detail, the method provides dynamic surgical feedback during a surgical or therapeutic procedure by encoding an integrated micromechanical device, such as a MEMS device, with appropriate power, sensing, and transmission capabilities, and disposing the integrated micromechanical device in a fluid path resulting from the therapeutic procedure. An external control or diagnostic system such as a fluid management system activates the integrated micromechanical device via a wireless signal for transmitting a return signal indicative of measured surgical parameters, and the control system receives the return signal for determining the measured surgical parameters.

In a particular configuration, the claimed approach has particular utility in an endoscopic procedure such as a knee joint surgery, discussed herein as an example application. In a medical device environment, the method of measuring surgical parameters includes identifying a surgical void responsive to receiving a fluid flow for a therapeutic procedure, such that the void is in communication with an endoscopic instrument for performing the therapeutic procedure. In the example shown, the surgical void is a skeletal joint region between articulated skeletal members (tibia and femur). An integrated micromechanical device (micromechanical device) is encoded with power, sensing, and transmission capabilities, in which the micromechanical device is adapted for nonintrusive attachment to the endoscopic instrument. A surgeon introduces the micromechanical device into the surgical void via the endoscopic instrument, and directs a fluid flow into the surgical void for maintaining a positive pressure and evacuating surgical material resulting from the therapeutic procedure. Surgical instruments dispose the micromechanical device in a fluid path of the therapeutic procedure via the endoscopic instrument. The fluid management system activates the micromechanical device for measuring surgical parameters, typically including at least one of pressure, flow and temperature of the fluid flow within the surgical void, and the management system or controller receives the measured surgical parameters via a wireless transmission from the micromechanical device

Alternate configurations of the invention include a multiprogramming or multiprocessing computerized device such as a multiprocessor, controller or dedicated computing device or the like configured with software and/or circuitry (e.g., a processor as summarized above) to process any or all of the method operations disclosed herein as embodiments of the invention. Still other embodiments of the invention include software programs such as a Java Virtual Machine and/or an operating system that can operate alone or in conjunction with each other with a multiprocessing computerized device to perform the method embodiment steps and operations summarized above and disclosed in detail below. One such embodiment comprises a computer program product that has a non-transitory computer-readable storage medium including computer program logic encoded as instructions thereon that, when performed in a multiprocessing computerized device having a coupling of a memory and a processor, programs the processor to perform the operations disclosed herein as embodiments of the invention to carry out data access requests. Such arrangements of the invention are typically provided as software, code and/or other data (e.g., data structures) arranged or encoded on a computer readable medium such as an optical medium (e.g., CD-ROM), floppy or hard disk or other medium such as firmware or microcode in one or more ROM, RAM or PROM chips, field programmable gate arrays (FPGAs) or as an Application Specific Integrated Circuit (ASIC). The software or firmware or other such configurations can be installed onto the computerized device (e.g., during operating system execution or during environment installation) to cause the computerized device to perform the techniques explained herein as embodiments of the invention.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a medical device environment suitable for use with configurations disclosed herein;

FIG. 2 is a flowchart of dynamic parameter sensing as disclosed herein;

FIG. 3 is a diagram of sensor deployment in the environment of FIG. 1; and

FIGS. 4-6 are a flowchart of endoscopic sensory arrangements during a surgical procedure.

### DETAILED DESCRIPTION

Depicted below is an example configuration of a medical device environment employing dynamic surgical fluid sensing as disclosed herein. In a particular arrangement, the proposed approach may employ a sensor on a cannula or other surgical instrument for capturing real-time data within the skeletal joint defining the surgical site. A stand alone sensor may also be placed or affixed within the joint for similar operation. Other uses include disposing a sensor in a tube transporting surgical fluids to and from the surgical site, or in a cassette assembly or enclosure that houses repetitive and/or disposable equipment employed in the procedure. The size and placement of the sensors allows the sensors to be used to detect real-time data in strategic locations during the surgical procedure, and allows the data to be employed by the logic of the fluid management system as well as the surgeon or clinician for making clinical judgments about the procedure.

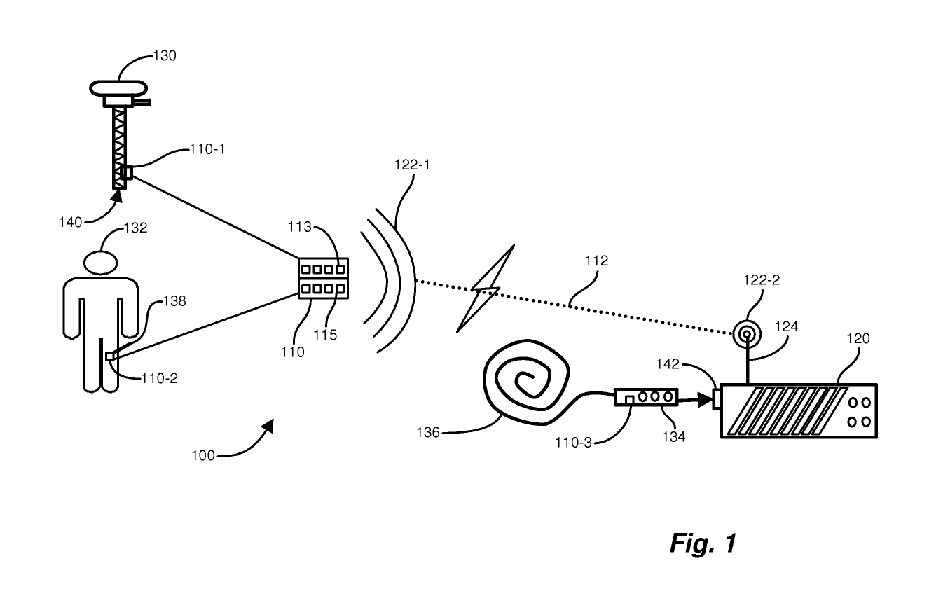

FIG. 1 is a context diagram of a medical device environment suitable for use with configurations disclosed herein. Referring to FIG. 1, a medical device environment 100 employs an integrated micromechanical device (micromechanical device) 110 for placement within the surgical environment. The micromechanical device 110, in a particular configuration, is a MEMS or NEMS device and maintains a wireless connection 112 to a fluid management system 120 or other centralized controller responsive to signals 122 to (122-1) and from (122-2) a wireless antenna 124. The micromechanical device 110 includes a receiver 115 responsive to the signals 122-2 from the antenna 124 for performing sensing surgical parameters, and a transmitter 113 configured to transmit the sensed surgical parameters back to the fluid management system 120 via signals 122-1. The micromechanical device 110 may be passive, such that the signals 122-2 also provide power to the sensor 110. The micromechanical device 110 is sufficiently small such that received signals 122-2 permit operation and transmission of sensed parameters 122-1, and the micromechanical device 110 may have other sensory areas, processing functions or mechanical features responsive to the signal 122-2.

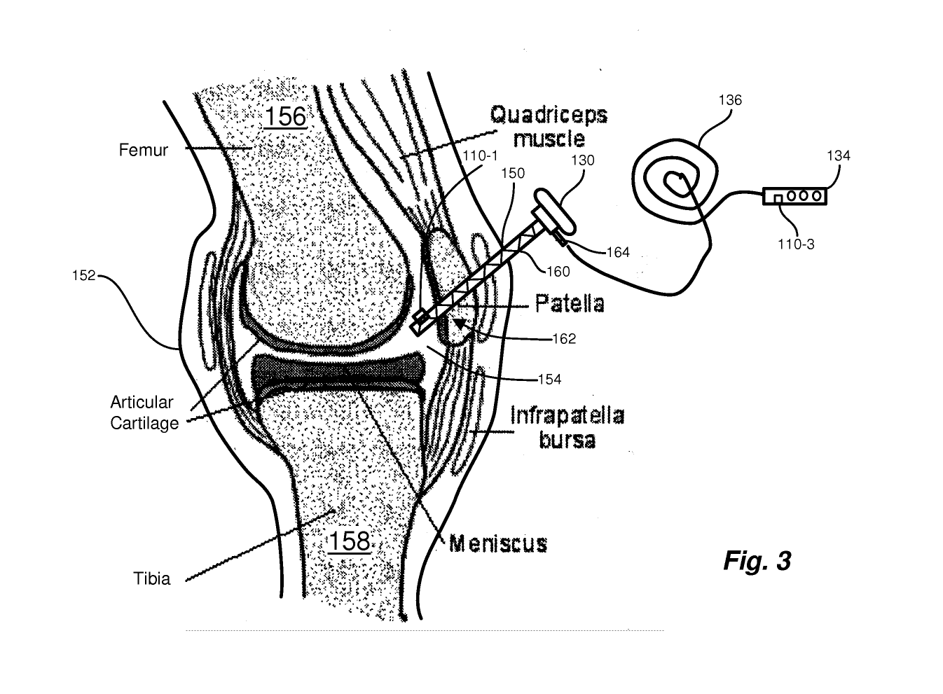

Placement of the micromechanical device 110 is such that it directly senses surgical parameters such as pressure, flow, and temperature, and may include affixation to the interior of a cannula 130, shown as micromechanical device 110-1, inserted in a surgical void or cavity of a patient 132, possibly via an endoscopic probe, shown as 110-2, or disposed (110-3) in a cassette 134 of a tube set 136 for pumping saline to a surgical site. The micromechanical device 110, once disposed, activates from a signal 122-2 from the fluid management system 120, and performs sensing, computation and transmission tasks for returning the sensed surgical parameters 122-1. The cannula 130 configuration affixes the micromechanical device 110-1 to the inside of a conduit 140 which is then inserted into a surgical void or cavity and saline delivered therethrough, discussed further below with respect to FIG. 3. A probe 138 arrangement allows disposition of the micromechanical device 110-2 through any suitable endoscopic orifice, and the cassette 134 based micromechanical device 110-3 is disposed within the cassette 134 in contrast to conventional approaches that employ a fragile transducer between the cassette 134 and a mating arrangement 142 on the fluid management system, which has been shown to be susceptible to repeated insertions.

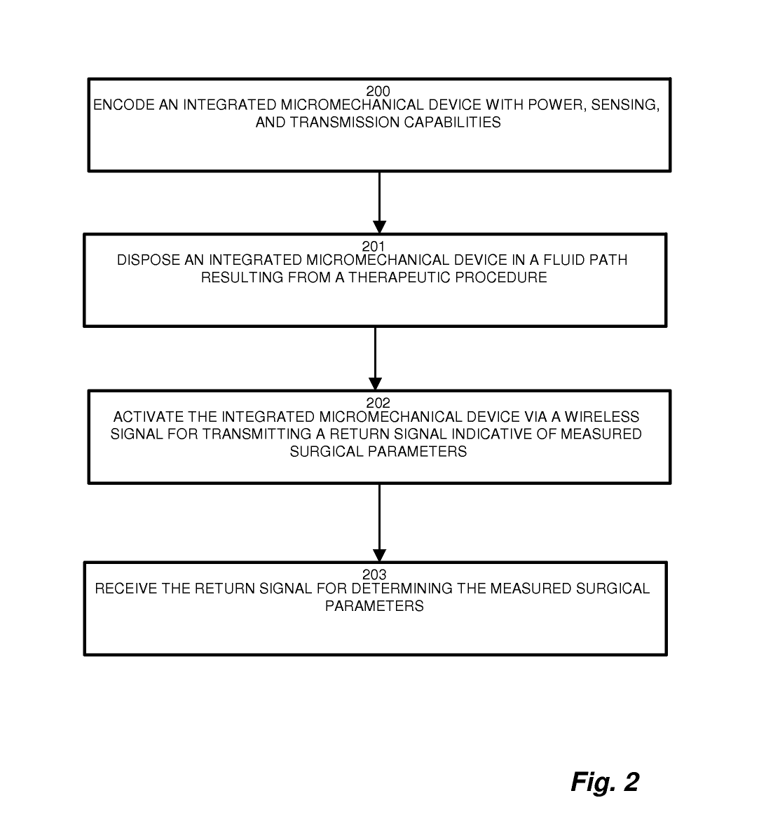

FIG. 2 is a flowchart of dynamic parameter sensing as disclosed herein. Referring to FIGS. 1 and 2, at step 200, the method of providing dynamic surgical feedback includes encoding an integrated micromechanical device with power, sensing, and transmission capabilities for gathering and returning sensory data. The method disposes the micromechanical device 110 in a fluid path resulting from a therapeutic procedure, as depicted at step 201. The micromechanical device 110 is a miniature machine such as a MEMS or NEMS structure and includes electronics for receiving processing and transmitting as well as physical structure for sensory and mechanical operations. A wireless signal 122-2 from the fluid manager 120 activates the integrated micromechanical device via an encoded transmitter 113/receiver 115 for transmitting a return signal indicative of measured surgical parameters, as disclosed at step 202, and the fluid manager 120 receiving the return signal 122-1 for determining the measured surgical parameters, as depicted at step 203. The measured parameters may include a variety of sensed attributes or characteristics from the surgical site, such as pressure resulting from a variable resistor sensor, flow relating to a baffle or fluid capture sensor, or temperature derived from a bi-metal sensor structure, for example.

FIG. 3 is a diagram of sensor deployment in the environment of FIG. 1. Referring to FIGS. 1 and 3, an example arrangement of micromechanical device 110 deployment in an endoscopic knee procedure is depicted. A surgeon disposes the cannula 130 through an endoscopic aperture 150 in the knee 152 of a patient. The cannula 130 extends through skin and soft tissue into the surgical void 154 between the femur 156 and tibia 158. The micromechanical device 110-1 affixed to the interior of a delivery tube 160 of the cannula 130 senses pressure, flow and temperature of saline pumped through the cannula delivery tube 160 by positioning in the fluid path at a delivery end 162 of the cannula 130. A supply nipple 164 attaches to the tube set 136 for supply the saline via the cassette 134 from the fluid management system 120. The cassette 134 may also include another micromechanical device 110-3 in the cassette 134 for sensing surgical parameters at the saline source when pumped from the fluid management system 120.

In the example shown, the integrated micromechanical devices 110-1, 110-3 are positioned in the fluid flow from the fluid management system 120 for directly sensing surgical parameters such as pressure, flow rate, and temperature. The micromechanical devices 110 may be disposed of with the cannula 130 and tube set 134 (single use items) following usage, thus low cost fabrication of the integrated micromechanical device 110 avoids prohibitive costs. In a particular arrangement, the improved accuracy by direct sensing in the surgical site avoids the need for additional medical devices for sensing the surgical parameters, thus maintaining or reducing the overall per-procedure cost of single use items. Alternative arrangements of the MEMS and NEMS devices 110 may be envisioned for affixation to other medical devices, such as a dedicated probe 138, on a second cannula for evacuating the surgical void 154, or with other native and introduced surgical fluids (i.e. medication, blood, etc.). In the example arrangement, the medical devices such as the cannula 130 and tube set 136 are single use or intermittent usage items, and are not intended or required to maintain disposed in the fluid flow longer than the intended procedure. Accordingly, fabrication as single use items mitigates production costs as the micromechanical devices need not withstand prolonged fluid exposure as permanently implanted items would.

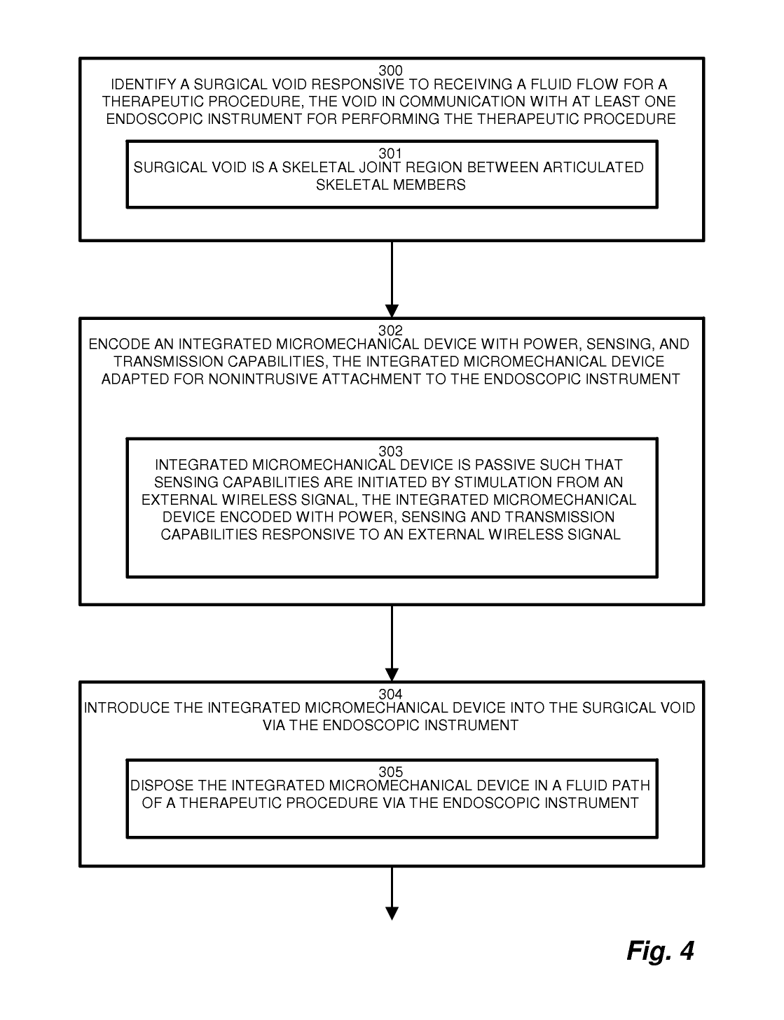

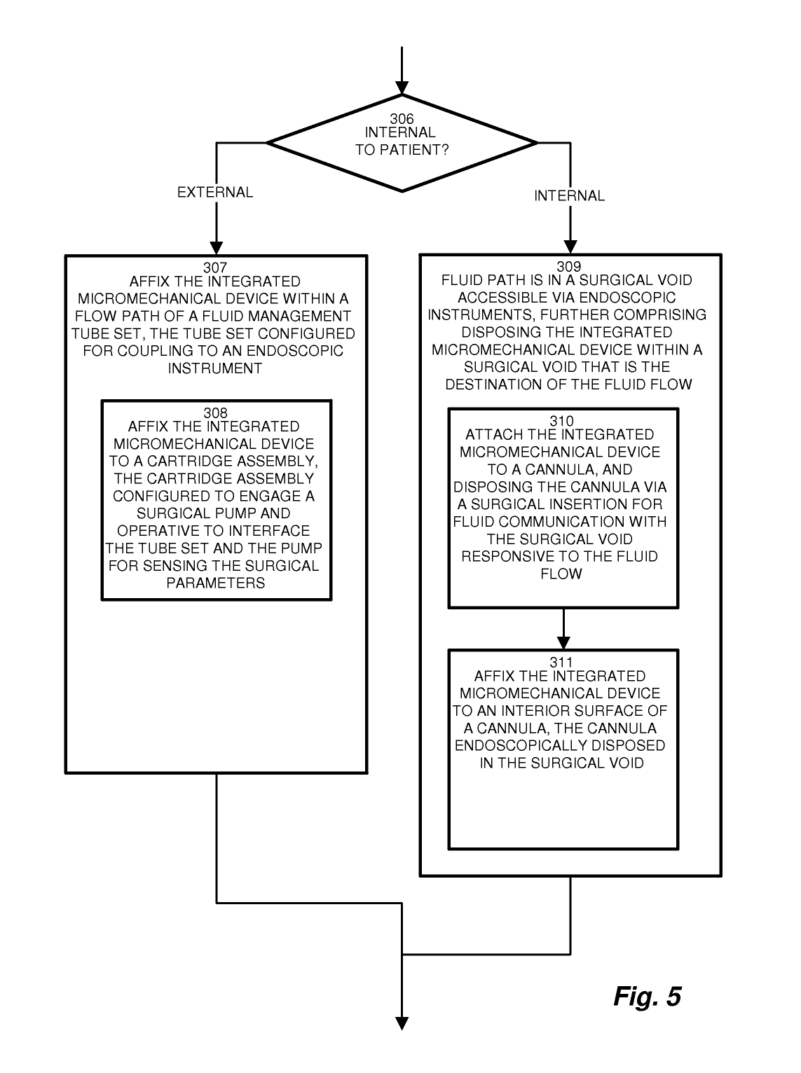

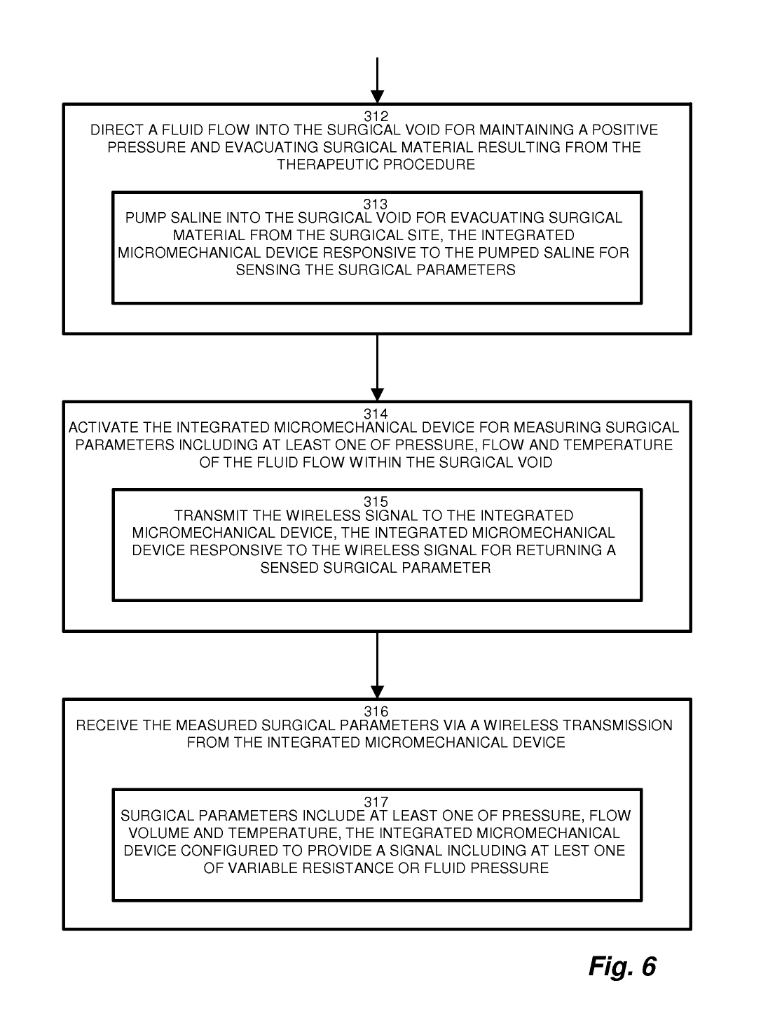

FIGS. 4-6 are a flowchart of endoscopic sensory arrangements during a surgical procedure. An example arrangement of an endoscopic surgical procedure on a knee joint 152 is shown, and employs a fluid management system 120 for delivering saline solution for irrigating the enclosed, internal joint region during surgery. Referring to FIGS. 1 and 3-6, In the medical device environment 100, the method of measuring surgical parameters as disclosed herein includes identifying a surgical void 154 responsive to receiving a fluid flow for a therapeutic procedure, in which the void 154 is in communication with at least one endoscopic instrument 130, 138 for performing the therapeutic procedure, as depicted at step 300. In the disclosed arrangement shown, the surgical void 154 is a skeletal joint region between articulated skeletal members (tibia 158 and femur 156), as shown at step 301. Other surgical voids or regions may employ similar surgical instruments. An initialization process encodes an integrated micromechanical device 110, such as a MEMS or NEMS device, with power, sensing, and transmission capabilities, such that the micromechanical device is adapted for nonintrusive attachment to the endoscopic instrument 1390, 138, as depicted at step 302. Various arrangements for coupling the micromechanical device 110 to a surgical or endoscopic instrument may be employed, as depicted below. Such a device 110 may be adhered or affixed to an interior annular surface or a pipe, tube or vessel carrying the surgical fluids, or may be attached to an exterior surface of a probe 138 inserted into the void 154 or surgical site. In particular arrangements, the integrated micromechanical device 110 may be passive such that sensing capabilities are initiated by stimulation from an external wireless signal 122-2, in which the micromechanical device 110 is encoded with power, sensing and transmission capabilities responsive to the external wireless signal 122-2, as depicted at step 303. Such devices 110 are sufficiently small that an RF control signal or other electromagnetic waveform is ample for the device 110 to draw operational power. Optionally, an active power source may be employed on the device 110, such as a battery element.

The endoscopic instrument on which the device 110 is affixed introduces the integrated micromechanical device 110 into the surgical void 154 via the endoscopic instrument 130, 138, as shown at step 304, typically through one or more of the surgical apertures 150 common to endoscopic, laparoscopic and other minimally invasive procedures. The endoscopic instrument 130, 138 is introduced into the void 154 for disposing the integrated micromechanical device 110 in a fluid path of a therapeutic procedure via the endoscopic instrument 130, 138, as shown at step 305.

A check is performed, at step 306, to determine if the micromechanical device 110 is disposed internally at the surgical site, or integrated in an external appliance or device. When the fluid path is in a surgical void accessible via endoscopic instruments, a probe 138 or cannula 130 disposes the integrated micromechanical device 110 within the surgical void 154 that is the destination of the fluid flow, as depicted at step 309. Disposing the micromechanical device 110 includes attaching the integrated micromechanical device to a cannula 130, probe 138, or similar surgical instrument, and disposing the cannula 130 via a surgical insertion 150 for fluid communication with the surgical void 154 responsive to the fluid flow, as disclosed at step 310. Epoxy, glue clips, or other attachment mechanism affixes the integrated micromechanical device 110 to an interior surface of a cannula 130, and the cannula 130 is endoscopically disposed in the surgical void 154, as depicted at step 311. The micromechanical device 110 directly senses surgical parameters, as the fluid characteristics in the enclosed, internal endoscopic surgical sit may vary from parameters sensed elsewhere in the fluid flow.

The disclosed approach may also include affixing the integrated micromechanical device within a flow path of a fluid management tube set 136, in which the tube set 136 is configured for coupling to an endoscopic instrument such as the cannula 130, as disclosed at step 307. The tube set 136 is often employed for transporting surgical fluids such as saline to a surgical site for irrigation, debridement, or maintaining a positive pressure in the surgical void 154 to maximize clearance for endoscopic instruments. Such configurations may further include affixing the integrated micromechanical device 110 to a cassette 134 or cartridge assembly, the cassette assembly configured to engage a surgical pump and operative to interface the tube set 136 and the pump for sensing the surgical parameters, as depicted at step 308. The cassette 134 is often employed for readily attaching and detaching the tube set 136 from the fluid management system 120, which includes the pump, to separate the fluid system (tube set) of one patient from the fluid management system 120 that is reused on multiple patients. Conventional approaches employ a transducer coupled to the cassette 134 assembly for capturing surgical parameters, however this transducer arrangement is fragile and prone to failure from repeated insertion of the cassette 134 in the fluid management system 120.

The fluid management system 120 directs a fluid flow into the surgical void 154 for maintaining a positive pressure and evacuating surgical material (debriding) resulting from the therapeutic procedure, as depicted at step 312. Typically this involves pumping saline into the surgical void 154 for evacuating surgical material from the surgical site, such that the integrated micromechanical device 110 is responsive to the pumped saline for sensing the surgical parameters, as shown at step 313. Due to the micromechanical nature of the device 110, its presence does not impede or adversely affect fluid flow, and the wireless interface avoids introduction of additional tethers (wires) into the surgical field.

The fluid management system 120 activates the integrated micromechanical device 110 for measuring surgical parameters including at least one of pressure, flow and temperature of the fluid flow within the surgical void, as disclosed at step 314. Activation includes transmitting the wireless signal 122-2 to the integrated micromechanical device 110, such that the integrated micromechanical device 110 is responsive to the wireless signal 122-2 for returning a sensed surgical parameter in a return wireless message 122-1, as depicted at step 315. In the case of a passive device, power requirements for operation of the micromechanical device 110 derive from the received signal 122-2, and commence sensing, computation and transmission of the surgical parameters.

The fluid management system 120 receives the measured surgical parameters via the wireless transmission 122-1 from the micromechanical device 110, as depicted at step 316 for usage by the fluid management system 120 as diagnostic feedback and control information. In the example arrangement, the surgical parameters include at least one of pressure, flow volume and temperature, such that the integrated micromechanical device 110 is configured to provide a signal based on at least one of variable resistance or fluid pressure sensed in the surgical void 154, as depicted at step 317. Other surgical parameters and sensed characteristics may be employed in alternate arrangements.

Conventional approaches are shown by U.S. Publication No. 2007/0007184, by Voto, for example, which shows a hemodialysis system having a disposable sensor combined with a dialysis circuit. The disposable sensor is either itself virtually or completely biochemically inert. In the proposed and claimed approach, the sensor is disposed within a surgical site, external to a blood vessel and not within a fluid path recirculating to the patient. Accordingly, Voto ‘184 differs from the proposed approach by sensors which are agnostic or non-reentrant to blood contact, such that the sensed fluid is not repetitively cycled back across the same sensor.

U.S. Publication No. 2010/0051552 (Rohde ‘552), assigned to K&L Gates LLP of Chicago, Ill., shows a system for monitoring water quality for dialysis, dialysis fluids, and body fluids treated by dialysis fluids. In Rohde ‘552, sensors are placed at various positions and are capable of detecting numerous properties and species in a variety of aqueous fluids including water, dialysis fluid, spent dialysis fluid and even blood. However, in contrast to the proposed approach, there is no showing, teaching, or disclosure of placement of MEMS or NEMS sensors within a surgical site such as a bone joint for monitoring fluid properties at a surgical site.

Varadan, U.S. Pub. No. 2006/0212097 discloses the use of MEMS technology in the treatment of Parkinson's disease (PD). A procedure known as Deep Brain Stimulation (DBS) is useful for treating tremor, dyskinesias, and other key motor features of PD. Varadan ‘097 teaches providing biocompatible materials for use in the microfabrication of implantable devices and systems Accordingly, the Varadan approach, employs a water soluble, non-toxic and non-immunogenic polymer such as Poly(ethylene glycol)(PEG)/poly(ethylene oxide) (PEO), a well-known polymer that can be used as a silicon coating for biological applications, for providing biocompatibility. As the proposed approach employs MEMS sensors for surgical procedures, long term implantation and corresponding biocompatibility is not required. The proposed approach, in contrast, employs temporary sensors in a fluid path for the duration of a surgical procedure, rather than long term brain implants requiring biocompatible materials for use in the microfabrication of implantable devices and systems.

Those skilled in the art should readily appreciate that the programs and methods for measuring surgical parameters as defined herein are deliverable to a user processing and rendering device in many forms, including but not limited to a) information permanently stored on non-writeable storage media such as ROM devices, b) information alterably stored on writeable non-transitory storage media such as floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and method of measuring surgical parameters has been particularly shown and described with references to embodiments thereof, it will be understood by those skilled in the art that various changes in form and details may be made therein without departing from the scope of the invention encompassed by the appended claims.

What is claimed is:

1. In a medical device environment, a method of measuring surgical parameters comprising:
identifying a surgical void responsive to receiving a fluid flow for a therapeutic procedure, the surgical void in communication with at least one endoscopic instrument for performing the therapeutic procedure;
encoding an integrated micromechanical device with power, sensing, and transmission capabilities, the integrated micromechanical device adapted for nonintrusive attachment to the endoscopic instrument;
introducing the integrated micromechanical device into the surgical void via the endoscopic instrument;
directing a fluid flow into the surgical void for maintaining a positive pressure and evacuating surgical material resulting from the therapeutic procedure;
disposing the integrated micromechanical device in a fluid path of a therapeutic procedure via the endoscopic instrument;
activating the integrated micromechanical device for measuring surgical parameters including at least one of pressure, flow and temperature of the fluid flow within the surgical void; and
receiving the measured surgical parameters via a wireless transmission from the integrated micromechanical device.

2. The method of claim 1 wherein the surgical void is a skeletal joint region between articulated skeletal members.

3. A method of providing dynamic surgical feedback comprising:
encoding an integrated micromechanical device with power, sensing, and transmission capabilities;
disposing the integrated micromechanical device in a fluid path resulting from a therapeutic procedure;
activating the integrated micromechanical device via a wireless signal for transmitting a return signal indicative of measured surgical parameters; and
receiving the return signal for determining the measured surgical parameters.

4. The method of claim 3 wherein the fluid path is in a surgical void accessible via endoscopic instruments, further comprising disposing the integrated micromechanical device within a surgical void that is the destination of the fluid flow.

5. The method of claim 4 wherein disposing includes attaching the integrated micromechanical device to a cannula, and disposing the cannula via a surgical insertion for fluid communication with the surgical void responsive to the fluid flow.

6. The method of claim 5 wherein activating further comprises transmitting the wireless signal to the integrated micromechanical device, the integrated micromechanical device responsive to the wireless signal for returning a sensed surgical parameter.

7. The method of claim 6 wherein the integrated micromechanical device is passive such that sensing capabilities are initiated by stimulation from an external wireless signal, the integrated micromechanical device encoded with power, sensing and transmission capabilities responsive to an external wireless signal.

8. The method of claim 4 further comprising pumping saline into the surgical void for evacuating surgical material from the surgical site, the integrated micromechanical device responsive to the pumped saline for sensing the surgical parameters.

9. The method of claim 8 wherein the surgical parameters include at least one of pressure, flow volume and temperature, the integrated micromechanical device configured to provide a signal including at least one of variable resistance or fluid pressure.

10. The method of claim 3 wherein disposing further comprises affixing the integrated micromechanical device within a flow path of a fluid management tube set, the tube set configured for coupling to an endoscopic instrument.

11. The method of claim 10 further comprising affixing the integrated micromechanical device to a cassette assembly, the cassette assembly configured to engage a surgical pump and operative to interface the tube set and the pump for sensing the surgical parameters.

12. The method of claim 10 further comprising affixing the integrated micromechanical device to an interior surface of a cannula, the cannula endoscopically disposed in the surgical void.

13. An apparatus for providing dynamic surgical feedback comprising:
an integrated micromechanical device encoded with power, sensing, and transmission capabilities; and
an affixation to a surgical instrument for disposing the integrated micromechanical device in a fluid path resulting from a therapeutic procedure;
the integrated micromechanical device including:
a receiver for activating the integrated micromechanical device via a wireless signal for transmitting a return signal indicative of measured surgical parameters; and
a transmitter for transmitting a return signal to a management system configured to receive the return signal for determining the measured surgical parameters.

14. The apparatus of claim 13 wherein the receiver is responsive to the transmitted wireless signal, the integrated micromechanical device responsive to the wireless signal for returning a sensed surgical parameter.

15. The apparatus of claim 14 wherein the integrated micromechanical device is passive such that sensing capabilities are initiated by stimulation from an external wireless signal, the integrated micromechanical device encoded with power, sensing and transmission capabilities responsive to an external wireless signal.

16. The apparatus of claim 14 further comprising an affixation to a surgical instrument employing a conduit for receiving pumped saline into the surgical void for evacuating surgical material from the surgical site, the integrated micromechanical device responsive to the pumped saline for sensing the surgical parameters.

17. The apparatus of claim 16 wherein disposing further comprises affixing the integrated micromechanical device within a flow path of a fluid management tube set, the tube set configured for coupling to an endoscopic instrument.

18. The apparatus of claim 14 further comprising an affixation to a cassette assembly for affixing the integrated micromechanical device, the cassette assembly configured to engage a surgical pump and operative to interface the tube set and the pump for sensing the surgical parameters.

19. The apparatus of claim 14 further comprising an affixation to an interior surface of a cannula for affixing the integrated micromechanical device to the cannula endoscopically disposed in the surgical void via a surgical insertion for fluid communication with the surgical void responsive to the fluid flow.

20. In a medical device environment, a non-transitory computer readable storage medium having logic encoded as instructions that when executed by a processor responsive to the instructions, perform a method of dynamic sensing of surgical parameters, the method comprising:
encoding an integrated micromechanical device with power, sensing, and transmission capabilities;
disposing the integrated micromechanical device in a fluid path resulting from a therapeutic procedure;
activating the integrated micromechanical device via a wireless signal for transmitting a return signal indicative of measured surgical parameters; and
receiving the return signal for determining the measured surgical parameters.
